(19) United States
(12) Patent Application Publication
(10) Pub. No.: US 2025/0003844 A1
(43) Pub. Date: Jan. 2, 2025

(54) METHOD FOR CASCADABLE CONCENTRATION OF AT LEAST ONE TARGET SUBSTANCE IN A SAMPLE LIQUID

(71) Applicant: IST Innuscreen GmbH, Berlin (DE)

(72) Inventors: Timo Hillebrand, Hönow (DE); Elmara Graser, Berlin (DE); Wiebke Jacobi, Falkensee (DE)

(21) Appl. No.: 18/707,767

(22) PCT Filed: Nov. 18, 2022
(86) PCT No.: PCT/EP2022/082367

§ 371 Date: May 6, 2024

(30) Foreign Application Priority Data
Nov. 22, 2021 (DE) 10 2021 130 537.3
Nov. 24, 2021 (DE) 10 2021 130 831.3
Dec. 7, 2021 (DE) 10 2021 132 210.3
Dec. 7, 2021 (DE) 10 2021 132 214.6
Feb. 15, 2022 (DE) 10 2022 103 554.9

(51) Int. Cl. G01N 1/40

(52) U.S. Cl. CPC G01N 1/405; G01N 1/4077; G01N 2001/4088

(57) ABSTRACT

A method for concentrating at least one target substance in a sample liquid comprises adding a superabsorber to an initial volume of liquid of the sample liquid or adding the volume of liquid to the superabsorber, incubating, over a first period, the mixture obtained by mixing the superabsorber and the volume of liquid, and removing a first sample of the liquid portion of the mixture present after incubation.

The invention relates to a method for cascadable concentration of at least one target substance in a sample liquid.

The detection of target substances, in particular of biological target substances, plays an important role in liquid analysis. Target substances to be detected or monitored can be biomolecules, such as eukaryotic cells, prokaryotic cells, subcellular vesicles, bacteriophages, viruses, toxins, antibodies, or also nucleic acids or proteins. Analysis methods are used for the qualitative or quantitative determination of target substances in samples taken from a liquid to be analyzed. For this purpose, laboratory methods that can be carried out automatically and/or at least partially automatically are available, as are analyzers operating in a completely automated manner. The analyzers operating in an automated manner also include online analysis devices which continuously or discontinuously remove samples from the liquid to be monitored and carry out a qualitative or quantitative determination of the target substance.

At low concentrations of the target substance in the liquid to be analyzed, the problem arises during sampling of providing a sufficient amount of the target substance for the subsequent analysis. In some applications, the target substance is present in an untreated liquid sample at a concentration which is too low for the subsequent processing or analysis. This results in the need to concentrate the target substance in the sample. An example of this is the detection of biomolecules in water or wastewater, for example the detection of SARS-COV-2 in water by means of molecular genetic techniques such as PCR or real-time PCR. The concentrations of the virus particles to be detected or of viral fragments in the wastewater are often too low to detect them by means of the methods known to a person skilled in the art. If a volume of 200 μL is sufficient when examining blood samples for the presence of a viral infection, the required sample volume is significantly greater during the analysis of water/wastewater. The literature describes starting amounts of up to multiple liters.

The concentration of target molecules in a volume of liquid plays an important role not only for the preparation of samples for molecular genetic analysis techniques, but equally also for all immunological technologies and spectroscopic technologies, such as molecular spectroscopy or mass spectroscopy, for example.

In the prior art, various techniques are known for the enrichment of viruses or subcellular particles from a biological sample, for example ultracentrifugation techniques or the technology of ultrafiltration. These methods are time-consuming and relatively expensive. Alternative methods consist of precipitation of virus particles by means of polyethylene glycol/sodium chloride and subsequent centrifugation (Yamamoto et al., Virology 40 (1970) 734; Morandi et al., J. Clin. Microbiol. 36 (1998) 1543-1538). Various mixtures of PEG and sodium chloride are used, and these reagents mix with the biological sample. Subsequently, the batch is incubated under refrigeration for a longer period, and subsequently the virus-(or protein)-NaCl/PEG precipitates are obtained by centrifugation. These methods are also complicated and require a lot of time. Furthermore, the further processing of the precipitates to isolate the viral nucleic acids is problematic. The precipitates can often be brought into solution only with great difficulty. This considerably influences the efficiency and the quality of the nucleic acid isolation.

A disadvantage of these techniques is also that concentrating a large-volume sample is very complex in implementation—for example, by means of known ultrafiltration methods. This also relates to the method described in patent specification EP 2283026 B1. This efficiently allows the processing of sample volumes of 5-10 mL, but is less efficient with larger sample volumes.

US 2015/0224502 A1 discloses a sample collection device for flow-through sampling in bodies of water. Here, for concentration of target substances present in bodies of water at low concentration, the route is taken to retain the target substances in a filter or adsorption medium, and to release the substances adsorbed on the filter by elution or as a lysate for subsequent analysis, and to provide them to an analysis module. This method is also complex in terms of apparatus and cannot be used universally.

The object of the invention is to provide a simple, rapid and universally usable method for concentrating target substances, in particular biomolecules, in a sample liquid.

This object is achieved in a surprisingly simple and universally applicable manner by means of the method defined in claim 1 and the use according to claim 15. Advantageous embodiments are listed in the dependent claims.

The method according to the invention for concentrating at least one target substance in a sample liquid comprises:
adding a superabsorber to an initial volume of liquid of the sample liquid or adding the volume of liquid to the superabsorber,
incubating the mixture obtained by mixing the superabsorber and the volume of liquid over a first period; and
removing a first sample of the liquid portion of the mixture present after incubation.

During incubation, the superabsorber takes up liquid, in particular a polar constituent of the sample liquid, such as, for example, water or another polar solvent, forming a gel or hydrogel, so that the volume of the liquid portion of the mixture decreases during the first period. The length of the first period can be defined individually and variably and correlates with the reduction in the initial volume of liquid that takes place after the end of incubation.

Superabsorbers (also: superabsorbent polymers, SAP) are plastics which are capable of absorbing a multiple of their own weight of polar liquids. Polar solvents, such as, for example, water or aqueous solutions, are suitable as liquids for absorption by the superabsorber. When the liquid is absorbed, the superabsorber swells and forms a gel or hydrogel. Hydrogels can form all crosslinked polymers which are polar, for example polyacrylamide, polyvinylpyrrolidone, amylopectin, gelatin, cellulose. However, plastics, in particular the plastics mentioned here and below, are preferred compared to biological polymers for the present invention.

Suitable for the invention is, for example, a copolymer of acrylic acid (propanoic acid, H₂C═CH—COOH) or sodium acrylate (sodium salt of acrylic acid, H₂C═CH—COONa) on the one hand, and acrylamide on the other hand, wherein the ratio of the two monomers to one another can vary. In addition, in the preparation of the copolymer, a so-called core crosslinker (CXL) can be added to the monomer solution, which connects (cross-links) the long-chain polymer molecules to one another by chemical bridges in some places. The polymer is water-insoluble because of these bridges. This so-called base polymer is optionally subjected to a so-called surface cross-linking (SXL). A further chemical is applied to the surface of the particles, which, by heating, links a second network only on the outer layer of the grain. This sheath supports the swollen gel, in order to hold it together even with external loading (movement, pressure).

Superabsorbers in the form of granules are used, for example, conventionally in baby diapers, monthly hygiene products, in incontinence care, and in wound dressing material. The use in cable sheathing for deep-sea lines is also known. Furthermore, superabsorbers are used as gel-forming extinguishing agents in fire control, as mechanical stabilizers for cut flowers in a vase, or as an additive for potting soil for long-term water storage. Because of its better environmental compatibility, potassium lye-neutralized acrylic acid is used here. In the form of spherical particles, the use of superabsorbers as a toy is known, with designations such as “Wasserperlen,” “Aqua Beads” or “Water beads.” These are superabsorbers which are commercially available in the form of beads of variable sizes of submillimeters to centimeters.

In the method according to the invention indicated above, the first sample, which is removed from the liquid portion of the mixture of liquid and superabsorber, which is present after incubation, can be the entire remaining liquid portion. However, it is also possible to remove only a partial volume of the given liquid portion as the first sample.

The first sample can be used directly for a subsequent analysis, for example by means of molecular genetic analysis technology. However, it can also be further concentrated in a cascading process in one or more additional stages, for example by readdition of a superabsorber and/or readdition to a superabsorber and reincubation, or by means of a conventional method for concentrating target substances, for example by one of the methods specified at the outset. If only a part of the liquid portion is removed as the first sample, the target substance in the liquid portion of the mixture remaining after the sample removal can be further concentrated by reincubation over a second period. Both variants of the method can be carried out repeatedly, so that a higher concentration of the target substance is obtained at each stage of the cascading concentration.

It is advantageous, in a first stage, to reduce the initial sample volume by means of the method according to the invention using a superabsorber, and in a subsequent second stage to carry out further concentration of the target substance by means of a conventional concentration technique, for example filtration, ultrafiltration, precipitation reaction, ultracentrifugation or coating by means of the method described in EP 2283026 B1. These known techniques can be used significantly more efficiently than in more strongly diluted solutions in already reduced sample volumes. The method according to the invention is thus suitable for significantly simplifying known methods for concentrating biomolecules for large-volume sample liquids and/or sample liquids containing low concentrations of the target substance.

As mentioned, in an advantageous embodiment, after removal of the first sample, the method can comprise a further concentration of the target substance in the removed first sample.

The further concentration of the target substance in the removed first sample can be carried out by means of a filtration, ultrafiltration or precipitation reaction technique.

Alternatively, however, the further concentration of the target substance in the removed first sample can also be carried out again, and optionally in a cascading manner, once or multiple times by carrying out the following method steps:
adding a superabsorber to the first sample or adding the first sample to the superabsorber,
incubating, over a second period, the mixture obtained by mixing the superabsorber and the first sample, and
removing a concentrated first sample of the liquid portion of the mixture present after incubation.

As described above in the first stage of the cascade, the concentrated first sample that is removed from the liquid portion remaining after incubation can comprise the entire volume of the liquid portion. Alternatively, the concentrated first sample can be a partial volume of the remaining liquid portion.

The target substance can be further concentrated in the concentrated first sample or in a further concentrated first sample obtained by further concentration, in particular by means of a superabsorber, by means of a filtration, ultrafiltration or precipitation reaction. This is advantageous if the volume of the concentrated first sample only corresponds to a few milliliters, e. g., 1 to 10 mL.

As already mentioned, the method according to the invention for concentrating the target substance can comprise the following further steps after removal of the first sample from the remaining liquid portion of the mixture from the originally used volume of liquid and the superabsorber:

optionally cascading, and singly or multiply repeated, reconcentrating the target substance in the liquid portion of the mixture remaining after removal of the first sample, by
reincubating, over a third period, the mixture remaining after removal of the first sample from the remaining liquid portion and the superabsorber; and
removing a second sample of the liquid portion of the mixture present after reincubation.

As a result of the reincubation, the volume of the liquid portion is further reduced, and accordingly the target substance is further concentrated in the liquid portion. The concentration of the target substance is determined, inter alia, by the prespecifiable duration of the third period and the conditions prevailing during the incubation.

In the method according to the invention, the initially used volume of liquid can contain a polar liquid, in particular as a main constituent. In an advantageous embodiment of the method according to the invention, the volume of liquid can contain a polar solvent, in particular as a main constituent. For example, the volume of liquid can consist of a polar solvent, at a mass fraction of at least 50%. The polar liquid or the polar solvent can be water, for example.

The target substance can be a biomolecule. It can be selected from the following substances: eukaryotic cells, constituents of eukaryotic cells, prokaryotic cells, constituents of prokaryotic cells, subcellular vesicles, bacteriophages, viruses, or virus constituents, toxins, antibodies, nucleic acids, and proteins.

As mentioned, in an advantageous embodiment, the superabsorber can be a plastics material or comprise a plastics material which absorbs a portion of the volume of liquid, for example a polar solvent contained in the volume of liquid, such as water, thereby forming a gel or hydrogel. Advantageously, the plastics material is selected such that it does not absorb biomolecules. This is the case, for example, in the above-mentioned superabsorbers made of the mentioned polymer and/or copolymer materials, for example, the commercially available Wasserperlen, Water Beads, etc.

The superabsorber can be used in the form of particles, for example as a powder, as granules, or in the form of geometric bodies, in particular spheres (spherical particles). It can thus be added to the volume of liquid and/or the first sample in the form of such particles, or the volume of liquid and/or the first sample can be added to the superabsorber in this form. The particles or spheres can have a diameter between 100 to 5000 μm.

Advantageously, the superabsorber is commercially available superabsorber spheres—for example, superabsorber beads available under the names “Aquabeads,” “Water Beads,” “Wasserperlen,” “Aquaperlen,” “Hydrokugeln,” “Gelkugeln.”

In an advantageous method embodiment, the volume of the liquid portion remaining after the step of incubating, and thus the concentration of the target substance in the remaining liquid portion, is controlled by the length of the period or the periods of incubation, by the size and number of the superabsorber particles or superabsorber spheres added to the initially used volume of liquid of the sample liquid and/or of the first sample, and/or by the temperature prevailing during the incubation.

The invention also comprises a use of a superabsorber for concentration, carried out once or multiple times in a cascading manner, in a liquid sample, of a target substance, in particular a biomolecule. The liquid sample can contain a polar liquid, in particular water, wherein the superabsorber is designed to absorb the polar liquid, for example water, or at least a portion of the polar liquid, thereby forming a hydrogel. The superabsorber can be formed from the materials described above and can be used in the above-described embodiments as granules, or—particularly preferably—in the form of spheres, in particular commercially available Wasserperlen, Water Beads or Aqua Beads.

The cascading concentration can comprise several stages. After each stage, a sample can be removed for analysis and the process of concentration can be continued, or the sample concentrated in at least one stage after incubation of the sample with the superabsorber or a portion of the sample can subsequently be further concentrated using other known methods.

The invention also comprises a kit for carrying out the method described above. The kit can comprise, for example, one or more containers with a pre-placed superabsorber, into which a user can then add an initial volume of liquid for concentration or a sample already concentrated in a first stage.

The liquid concentrated according to the described method and/or according to the described use, i.e. the liquid portion present after incubation, or a sample removed from the concentrated liquid can be supplied manually or automatically to a laboratory instrument for further treatment or analysis. In one possible embodiment of the method or the use, such a liquid or sample of the liquid can be added to a cartridge, in particular a microfluidic cartridge, of an automatic analysis device for the automated performance of a detection of the target substance by means of molecular genetic techniques such as PCR or real time PCR. This can take place manually or automatically.

The invention is explained in more detail below with reference to the drawings and several exemplary embodiments. These examples do not constitute any limitation of the agents and methods according to the invention.

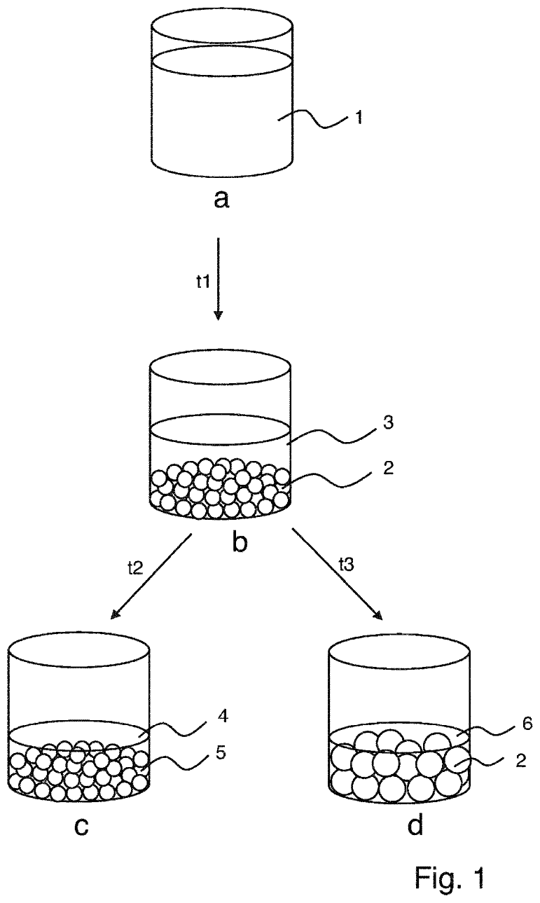

FIG. 1 is a schematic representation of the cascading concentration of a target substance in a liquid;
a) liquid before the addition of a superabsorber;
b) liquid after the addition of a superabsorber and incubating the mixture;
c) first sample removed from the mixture after adding a further superabsorber and incubating the mixture;
d) remaining mixture of liquid and superabsorber, optionally after removal of the first sample and after reincubation;

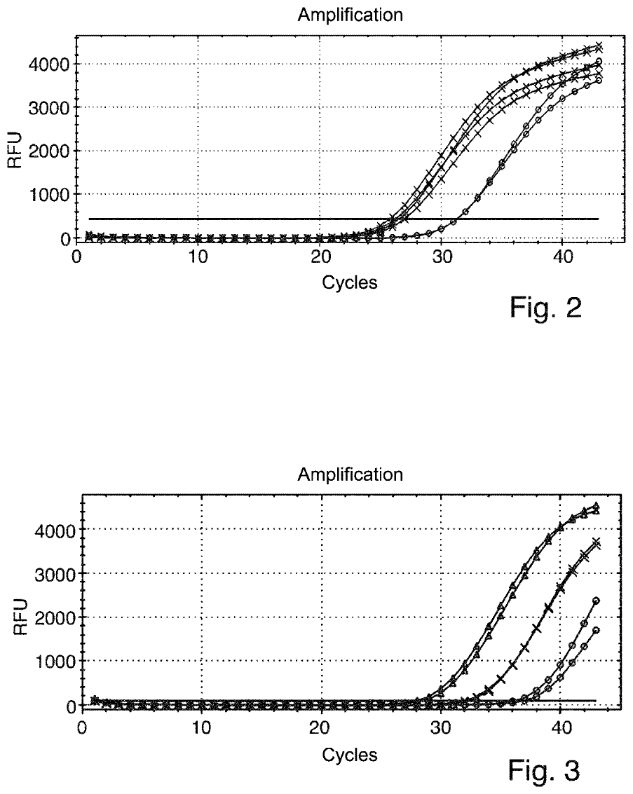

FIG. 2 shows amplification curves of different samples of water containing bacteriophages;

FIG. 3 shows amplification curves of different samples of phage-RNA-containing water.

The invention described here was based on the following unexpected observation. Commercially available so-called water beads (commercially available inter alia under the names Aqua Beads, Water Beads, Wasserbeads, or Gelperlen) were added to a volume of liquid of 1 liter. These water beads are made of a superabsorber material. The liquid was surface water which had been collected from an extinguishing water pond with suspended matter. After an incubation time, the water beads swell to a multiple of their original volume. The volume of the liquid portion of the mixture of the liquid and the water beads is reduced. Surprisingly, it was found that the suspended matter of the liquid was not absorbed by the swelling beads. The liquid portion of the mixture including the suspended matter (volume 400 mL) was transferred into a new vessel. A sample with a volume of 50 mL was taken from this liquid portion.

A comparison sample with a volume of 50 mL was removed directly from the liquid, i.e. the mentioned surface water, without the liquid previously being concentrated according to the method described. Both samples were centrifuged at 5000×g for 10 min. The supernatant was removed and the sediment was used for a nucleic acid extraction. The nucleic acid extraction was effected by means of a commercial kit (innuprep Stool DNA Mini Kit; IST Inuscreen GmbH). The DNA of both samples was then examined by real time PCR for the total bacterial germ count.

It was found that fewer bacteria were detected in the untreated comparative sample than in the sample concentrated with the superabsorber. This means that the bacteria contained in the liquid were not absorbed into the water beads. They were concentrated and were a constituent of the remaining liquid portion of the mixture of the liquid and the superabsorber spheres. These observations could also be confirmed below for other biomolecules, for example viruses or eukaryotic cells. After addition of the superabsorber to a volume of liquid containing these biomolecules, the volume was reduced and the biomolecules were concentrated in the remaining residual liquid. It was even more surprising that the method described can also concentrate proteins and free genomic DNA which are present in low concentrations in an aqueous solution.

The use of superabsorbers for concentrating a target substance, in particular biomolecules, in a polar liquid as solvent, for example water, for concentrating the target substance in the liquid, is very simple and universally applicable. A suitable method is described briefly with reference to FIGS. 1a and b as follows:
1. Addition of a superabsorber 2 to a volume of a liquid 1, in particular an aqueous liquid, or alternatively: addition of the liquid 1 to a superabsorber 2;
2. Incubation of the mixture of the liquid 1 and the superabsorber 2 over a first period t1 for reducing (reduction in volume) the liquid portion 3 of the mixture; and then
3. Transferring at least one first sample 4 of the liquid portion 3 of the mixture into a new vessel as a sample for further processing.

The further processing can be, for example, a nucleic acid extraction, a measurement, and/or a direct analysis with a wide variety of technologies, such as NGS applications, immunological technologies, spectroscopic technologies, molecular spectroscopy, or mass spectrometric technologies, etc.

The degree of concentration and the speed of this process can be controlled very precisely by means of the type of superabsorber used, by means of the amount thereof used, or by means of the incubation time and/or the incubation temperature.

With the method, the problem of processing low-concentration samples can thus be easily solved for a further processing of the target substances of interest, in particular biomolecules, or the detection thereof. The method shown in FIGS. 1a and b works without devices such as ultra-centrifuges, expensive ultrafiltration membranes, complex processes such as PEG precipitation, or generally precipitation reactions for concentrating nucleic acids, etc. In addition, the method can be used universally in relation to the target substances, in particular for biomolecules. A further advantage is that the superabsorbers are non-toxic and safe and often also biodegradable. The method according to the invention makes it possible to greatly simplify the analysis of low-concentration biomolecules.

In the case of large-volume and/or strongly diluted liquids which contain the target substance at a very low concentration, a cascading concentration of the target substance is possible. For example, in a first stage, the described method can be used with the above-mentioned steps 1-3 in order to concentrate the target substance. In a second stage, the volume of the first sample 4 can be reduced for reconcentration of the target substance. This can be done either by means of a conventional filtration or precipitation method, or other conventional methods. Alternatively, however, reconcentration of the target substance in the first sample 4 can also take place, as shown in FIG. 1 c, by adding fresh superabsorber 5 to the first sample 4, and reincubating over a second period t2. Additionally or alternatively, the liquid portion 6 remaining in a mixture with the superabsorber 2 after removal of the first sample 4 can be further reduced by incubating the mixture over a third period t3, as shown in FIG. 1d. This leads to further swelling with an increase in volume of the spheres consisting of the superabsorber 2, and to a further volume reduction in the liquid portion 6, which is accompanied by an increase in concentration of the target substance in the liquid portion 6.

In both alternative method paths, further cascading concentration stages can follow.

Some exemplary embodiments of the invention are described in more detail below.

### Exemplary Embodiment 1: Two-Stage Concentration of Bacteriophages from a Water Sample of 10 mL (Combination of Two Different Concentration Methods)

As the sample liquid, tap water in a volume of 10 ml was mixed with 5 μl of a bacteriophage suspension (Leibniz Institute DSMZ: DSM 13767). Two volumes of 10 mL each of the sample liquid were each transferred into a 50 mL vessel. 200 μL of the initial sample liquid were each taken as reference samples (reference samples 1 and 2) for the subsequent nucleic acid extraction. In a first concentration step, 40 superabsorber spheres were added to each 50 mL sample vessel. These are commercially available under the name “Water Beads” (approx. 1 mm diameter). The 10 mL sample volumes were concentrated to a volume of 1 mL by means of this step. This remaining liquid portion was removed in each case as a first sample and transferred into a 1.5 mL reaction vessel. The further concentration was carried out in this case by means of a known method (patent specification EP 2283026 B1). After addition of alginate and calcium chloride and a short incubation, the samples were each centrifuged at 13,000×g for 5 min. The supernatant was removed and the resulting pellet was resuspended with 200 μL 1×PBS. By combining the two methods, the initial sample of 10 mL could be concentrated to a volume of 200 μL. Both the 200 μL of the reference samples 1 and 2 and the two samples treated with the method according to the invention were then used for a subsequent nucleic acid extraction.

The nucleic acid extraction was carried out by means of an automated method on a KingFisher Flex (Thermo Fisher) machine with a commercially available kit (deltaPREP AniPath DNA/RNA Kit KFFLX; IST Inuscreen GmbH). The extracted phage RNA was used to detect the MS2 phage RNA using real time PCR. A commercial kit (innuDETECT Internal Control DNA/RNA Assay; IST Innuscreen GmbH) was used as the detection system. For the reverse transcription and amplification of the MS2 phage RNA, a commercial OneStep-RT-MasterMix was used (innuDRY qRT-PCR MasterMix Probe; IST Innuscreen GmbH). Two real time PCR runs (samples 3 to 6) were carried out for each sample.

FIG. 2 shows the amplification curves of the samples. The curves (circles) show the profiles of the amplification curves of the reference samples 1 and 2 of the sample liquid which was not reduced. The curves (dash and cross) are the amplification curves of the samples 3 to 6 of the sample liquid reduced according to the method according to the invention.

Table 1 indicates the Ct values for each sample.

TABLE 1

| Sample | Ct value |
|---|---|
| 1 (without concentration) | 31.28 |
| 2 (without concentration) | 31.26 |
| 3 (after two-stage concentration) | 26.22 |
| 4 (after two-stage concentration) | 25.78 |
| 5 (after two-stage concentration) | 26.81 |
| 6 (after two-stage concentration) | 26.59 |

The differences between the Ct-values show a calculated, about 50-fold concentration of the starting sample with respect to the initial amount of bacteriophages. This corresponds to the concentration of the sample liquid from an initial volume of 10 mL to a final sample volume of 200 μL (50 times reduction in volume).

### Exemplary Embodiment 2: Detection of MS2 Phase RNA after Cascading Concentration of an Initial Water Sample from 0.5 Liters to Different Concentration Stages

0.5 L of tap water dosed with MS2 bacteriophages was used as the sample liquid, and 0.5 L of water and 5 μL of an MS2 bacteriophage solution (Leibniz Institute DSMZ: DSM 13767) was added as a spike.

From the initial sample of 0.5 L, two samples (reference sample 1 and 2) each of 200 μL were taken as comparative samples for later nucleic acid extraction before cascading concentration. The first stage of the concentration cascade was carried out in such a manner that 10 g of superabsorber spheres, commercially available under the designation “Water Beads” (diameter: about 1 mm) were added to the 0.5 L water sample (in a 1 liter bottle). After a first incubation time of about 1.5 h, the residual volume of the liquid portion of the mixture was 50 mL. 200 μL each of these 50 mL of the concentrated sample liquid were removed (sample 3 and sample 4). The remaining liquid content of the mixture with the volume of about 50 mL was then transferred completely as a sample from the 1 liter bottle into a 50 mL reaction vessel for a second stage of the concentration cascade. 1.5 g of new “Water Beads” (diameter: about 1 mm) were added into the sample. The sample was incubated with the beads until the volume was reduced to 5 mL. The 5 mL was then transferred into a 15 mL reaction vessel, and 200 μL of sample were again taken from each of the 5 mL (sample 5 and sample 6). By means of the method according to the invention, in the first concentration stage, the sample was thus concentrated from 500 mL to 50 mL (factor of 10), and in the second concentration stage from 50 mL to 5 mL (factor of 10). Both cascading stages yielded a concentration of the sample from 500 mL down to 5 mL (factor of 100).

The phage-RNA extraction was carried out by means of an automated method on a KingFisher Flex (Thermo Fisher) machine with a commercially available kit (deltaprep AniPath DNA/RNA Kit KFFLX; IST Inuscreen GmbH). The extracted phage RNA was used to detect the MS2 phage RNA using real time PCR. A commercial kit (innuDETECT Internal Control DNA/RNA Assay; IST Innuscreen GmbH) was used as the detection system. For the reverse transcription and amplification of the MS2 phage RNA, a commercial OneStep-RT-MasterMix was used (innuDRY qRT-PCR MasterMix Probe; IST Innuscreen GmbH).

FIG. 3 shows the amplification curves of samples 1 to 6. The curves (circles) show the profiles of the amplification curves of the reference samples 1 and 2 of the sample liquid which was not reduced. The curves (cross) are the amplification curves of samples 3 and 4 (1st cascade), and the curves (triangle) are the amplification curves of samples 5 and 6 (2nd cascade).

Table 2 indicates the Ct values for the individual samples:

TABLE 2

| Sample | Ct value |
|---|---|
| 1 (without concentration) | 35.93 |
| 2 (without concentration) | 36.73 |
| 3 (following the 1st cascade, | 31.72 |
| concentration factor of 10) | |
| 4 (following the 1st cascade, | 32.07 |
| concentration factor of 10) | |
| 5 (following the 2nd cascade, | 28.23 |
| concentration factor of 100) | |
| 6 (following the 2nd cascade, | 27.68 |
| concentration factor of 100) | |

Here too, the data impressively show the effect of concentration both with respect to the initial samples and within the two cascading stages.

### Exemplary Embodiment 3: Measurement of the Change in Concentration of a DNA Solution Over Time Using the Method According to the Invention

The embodiment is meant to demonstrate that an analyte can be concentrated to any desired degree by means of the method according to the invention.

200 μL of a lambda DNA solution were used as the sample liquid. The initial concentration of the DNA was 132 ng/μL. The concentration was carried out by the addition of a superabsorber sphere, commercially available under the designation “Water Beads” (0.006 g; approx. 1 mm diameter). The concentration was determined every 10 minutes after incubation start in each case for 10 minutes in the form of spectrophotometric measurement of the sample. The volume of the liquid portion was reduced continuously over the incubation time. The DNA was concentrated in the solution in a cascading manner in 5 stages (measurement after 10 minutes, 20 minutes, 30 minutes, 40 minutes, and 50 minutes). After incubation for 30 minutes, the volume of the liquid portion was about 70 μL, and the DNA concentration had increased to 335 ng/μL.

The results of the concentration measurements are summarized in Table 3. The data impressively show the increase in the concentration of the DNA as a function of time. It is thus possible for the user to adjust a desired concentration of an analyte by suitably selecting the incubation time and the number of concentration stages carried out in a cascading manner. For this purpose, after a desired incubation time, the user would terminate the incubation with the superabsorber and remove the sample from the superabsorber for further use.

TABLE 3

| | DNA |
|---|---|
| Sample | concentration |
| 1 sample liquid before concentration | 132 ng/μL |
| 2 (cascade 1; 10 min incubation) | 142 ng/μL |
| 3 (cascade 1; 20 min incubation) | 189 ng/μL |
| 4 (cascade 1; 30 min incubation) | 335 ng/μL |
| 5 (cascade 1; 40 min incubation) | 451 ng/μL |
| 3 (cascade 1; 50 min incubation) | 810 ng/μL |

1-18. (canceled)

19. A method for concentrating at least one target substance in a sample liquid, comprising:
adding a superabsorber to an initial volume of liquid of the sample liquid or adding the initial volume of liquid to the superabsorber;
incubating, over a first period, a mixture obtained by mixing the superabsorber and the initial volume of liquid; and
removing a first sample of a liquid portion of the mixture present after incubation.

20. The method according to claim 19, further comprising:
further concentrating the target substance in the removed first sample.

21. The method according to claim 20,
wherein the further concentration of the target substance in the removed first sample is carried out by a filtration, an ultrafiltration, or a precipitation reaction technique.

22. The method according to claim 20,
wherein the further concentration of the target substance in the removed first sample is performed again by:
adding a superabsorber to the first sample or adding the first sample to the superabsorber,
incubating, over a second period, the mixture obtained by mixing the superabsorber and the first sample, and
removing a concentrated first sample of the liquid portion of the mixture present after incubation.

23. The method according to claim 22, further comprising:
concentrating the target substance in the concentrated first sample or in a further concentrated first sample obtained by further concentration.

24. The method according to claim 19, further comprising:
reconcentrating the target substance in the liquid portion of the mixture remaining after removal of the first sample by:
reincubating, over a third period, the mixture remaining after removal of the first sample from the remaining liquid portion and the superabsorber; and
removing a second sample of the liquid portion of the mixture present after reincubation.

25. The method according to claim 19,
wherein the volume of liquid contains a polar liquid.

26. The method according to claim 25,
wherein the target substance is a biomolecule selected from the group consisting of: eukaryotic cells, constituents of eukaryotic cells, prokaryotic cells, constituents of prokaryotic cells, subcellular vesicles, bacteriophages, viruses or virus constituents, toxins, antibodies, nucleic acids, and proteins.

27. The method according to claim 19,
wherein the superabsorber includes a plastics material which absorbs a portion of the volume of liquid, water, thereby forming a hydrogel.

28. The method according to claim 27,
wherein the plastics material does not absorb biomolecules.

29. The method according to claim 19,
wherein the superabsorber is used in the form of particles, granules, or geometric bodies.

30. The method according to claim 19,
wherein the superabsorber is used in the form of commercially available “Wasserperlen,” “Hydrokugeln,” “Aquaperlen,” “Aquabeads,” “Water Beads,” or “Gelkugeln.”

31. The method according to claim 24,
wherein the volume of the liquid portion remaining after incubation is controlled by the length of the period or periods of the incubation and/or by the type and/or amount of the superabsorber and/or by the temperature of the mixture prevailing during incubation.

32. The method according to claim 31,
wherein the superabsorber is used in the form of particles granules, or geometric bodies, and wherein the volume of the liquid portion remaining after incubation is controlled by the size and/or number of the particles.

33. A kit, comprising:
one or more containers with a pre-placed superabsorber, into which a user can then add an initial volume of liquid for concentration or a sample already concentrated in a first stage.
